(19) United States
(12) Patent Application Publication
(10) Pub. No.: US 2015/0119775 A1
(43) Pub. Date: Apr. 30, 2015

(54) STABILIZING ANKLE BRACE

(71) Applicant: DJO, LLC, Vista, CA (US)

(72) Inventors: Sean Gildersleeve, Carlsbad, CA (US); Charles Mogul, Mount Pleasant, SC (US); Richard Gildersleeve, Carlsbad, CA (US)

(21) Appl. No.: 14/066,069

(22) Filed: Oct. 29, 2013

(51) Int. Cl. A61F 5/01

(52) U.S. Cl. CPC A61F 5/0127
USPC 602/7

(57) ABSTRACT

Disclosed are ankle braces with heat moldable stays that provide inversion and eversion support. The stays include heat formable ankle regions to conform to the malleolus of the user's ankle.

### BACKGROUND

Certain ankle injuries, including severe ankle sprains, are common. Once injured, the ankle often becomes unstable for some period of time, and the risk of re-injury is increased. Moreover, repetitive sprains occurring over several years can result in long-term weakness.

Thus, it is desirable to have a brace that allows the user to move freely, while at the same time providing support during the recuperation period and providing long-term support to reduce the chance for re-injury.

A number of known orthopedic supports are available for treating injured parts of the anatomy. For example, some ankle braces have a plurality of pockets. Rigid stay members are inserted into the pockets and form a rigid structure that immobilizes the ankle.

A drawback of these designs is that the stays do not conform to the malleolus of the patient's ankle or account for variations in the patient's anatomy in the ankle region. Thus, the fit and support provided may not be satisfactory in some cases. Furthermore, the stays are typically die-cut from plastic and secured within the brace during manufacture. The shape of the stays is therefore quite limited, and they are typically not adjustable. The support often does not fit the anatomy adequately.

Improved designs are needed to stabilize the ankle against inversion and eversion.

### SUMMARY

Disclosed herein are ankle braces with one or more three-dimensional, reinforcing stays configured for stabilizing the ankle of a user against inversion and eversion. The ankle brace has an ankle support housing that is applied to the ankle of a user and can be tightened about the ankle by a fastener such as a strap or lace. The one or more stays have a supporting upper plate, for example plastic, having anterior and posterior edges and a proximal region that can be applied to a user, extending longitudinally along the user's leg above the user's ankle. The stay has an ankle region with an ankle-facing medial side and a non-ankle facing lateral side. The proximal region includes a contoured region that is disposed along the ankle-facing side to conform to the malleolus of the user's ankle. The contoured region is conformable by heat molding (or conformable by other process) and stiffens upon cooling to provide customized support for the user's ankle.

In one aspect, ankle braces are provided having a housing with an opening for receiving a user's ankle, lateral and medial sides that fit around the ankle, a compartment on at least one of the lateral and medial sides, and a stay sized to fit within the compartment. The compartment is a pocket, sleeve, or other receptacle disposed on a side of the brace housing. The pocket or other compartment has a proximal opening, and the stay is disposed within the compartment.

The stay includes a support plate having anterior and posterior edges, a distal region, and a proximal region that, when applied to a user, extends longitudinally along the user's leg above the user's ankle. The stay also includes a heat-moldable ankle region disposed longitudinally between the proximal and distal regions of the support plate, the heat-moldable ankle region having an ankle facing side, a non-ankle facing side, and a contoured region that is disposed along the ankle facing side and is positioned on the ankle-facing side so as to align with the ankle's malleolus.

The ankle brace includes at least one fastener, for example an exterior strap or a lace. The fastener is anchorable to the housing, for example by

Velcro or by tightening the lace, and is actuatable to tighten the brace about the ankle from a loose state to a tightened state. In certain embodiments, the support plate and ankle region are co-molded and heat moldable. In certain embodiments, the ankle region is a mid-region of the support plate that is co-molded to and unitary with the proximal and distal regions, such that the proximal, distal and mid-regions of the plate form a single contiguous, unitary plate that is heat moldable and stiffens upon cooling. In some embodiments, the stay is less than 0.06 inches thick. In some embodiments the thickness of the stay is between about 0.03 inches and about 0.06 inches thick, for example 0.04 inches.

In use, when the stay is in the loose state it is in a first shape configuration, and when the stay is in the tightened state it is in a second shape configuration that differs from the first shape configuration. The loose shape configuration is generally straight or slightly contoured, but the second shape configuration is customized to the patient in that it conforms to the specific contouring of the patient's external ankle region anatomy.

In certain embodiments, at least one of the anterior and posterior edges of the stay forms an anterior-posterior neck in the mid-region, which can provide added flexibility for dorsi and plantar flexion. The contoured region may be pre-formed within the stay prior to its insertion within the brace housing and application to the user. For example, the contoured region may include a cavity or crevice or series of spokes or ribs that are pre-formed within the stay prior to its insertion within the brace.

One or more strengthening features may also be included in the stay for added eversion/inversion support. In certain embodiments the contoured region comprises a plurality of ribs. In some implementations the ankle region includes a hub, and each of the plurality of ribs extends radially as a spoke about the hub. For example, the hub can be placed generally in the center of the ankle region, and the spokes extend radially about that center. In certain implementations, the plurality of ribs project from the ankle-facing side, for example in a medial direction toward the ankle.

In certain embodiments, the contoured region is a depression that is formed within the ankle-facing side prior to insertion of the stay in the pocket or sleeve. In certain embodiments, the contoured region of the support plate includes a first plurality of heat-moldable ribs that extend longitudinally between the anterior and posterior edges and proximal to the ankle region. At least one of the first plurality of ribs may extend longitudinally from a distal tip of the support plate to a proximal tip of the support plate. At least one of the first plurality of ribs may extend longitudinally through the ankle region.

In certain embodiments, a plurality of troughs are included within the medial side of the plate. The plurality of troughs are oriented on the support plate so as to extend longitudinally along the ankle-facing surface of the support plate. At least one of the first plurality of ribs is disposed between adjacent troughs. The plurality of troughs may extend longitudinally through the ankle region.

The ribs may also be adjusted so as to have a different configuration within the ankle region as compared to the distal or proximal regions. In certain implementations, the distal end of each rib of the first plurality of ribs terminates within or at a location proximal to the contoured region. One or more of a second plurality of ribs may also be used. In certain implementations, a second plurality of ribs is disposed on a medial side of the ankle region and forms a series of spokes around a hub in the ankle region, and at least one rib of the first plurality of ribs is spaced between adjacent ribs of the second plurality of ribs. At least one of the first plurality of ribs may extend longitudinally along the ankle region and the distal region of the plate.

In certain embodiments, adjacent ribs of the first plurality of ribs are spaced apart at their respective proximal ends by a first distance, and respective sections of those adjacent ribs are spaced apart within the ankle region by a second distance. The first distance may be greater than the second distance, such that the ribs converge toward each other in the ankle region. In certain implementations, the anterior and posterior edges of the support plate form a narrow neck in the ankle region. In certain embodiments, a second plurality of ribs extend longitudinally through the ankle region, the second plurality being fewer than the first plurality of ribs that extend along the ankle-facing surface of the proximal region. In certain implementations, one rib extends longitudinally through the ankle region, while a plurality of ribs are used in the proximal region.

In certain embodiments, the contoured region has a perimeter edge that is spaced between the anterior and posterior edges of the support plate. In certain embodiments, at least one of the anterior and posterior edges forms a flange in the mid-region. The flange extends in an anterior-posterior direction.

In another aspect, methods of assembling a brace and methods of use are included. A method of assembling an ankle brace using the techniques disclosed herein includes forming a pocket in a medial or lateral side of a flexible ankle housing, the pocket having an opening, forming a heat moldable stay having a width that fits within the opening, and inserting the stay into the pocket. The brace can then be applied to a patient for treating an ankle injury in a customized manner.

A method of treating the ankle injury includes inserting a heat moldable stay into a compartment (e.g., a pocket or sleeve) of an ankle brace, heating the stay-containing ankle brace to a temperature between about 150° F. and 250° F., inserting the ankle into the heated brace, and aligning the malleolus of the ankle with a medial side of the inserted stay. One or more fasteners (e.g., straps or laces) of the ankle brace are then tightened until the heated stay conforms to specific contours of the patient's ankle region anatomy.

The techniques provided herein can provide a number of advantages. For example, an injured ankle can be treated in a more customized manner. Braces can be provided that conform more specifically to the patient's ankle anatomy. The fit and comfort of the brace can be improved as the customized brace is applied. Inversion and eversion stiffness and dorsi and plantar flexion can also be more specifically modulated and controlled.

### BRIEF DESCRIPTION OF FIGURES

The above and other advantages of the brace structures and methods disclosed herein will be more apparent upon consideration of the following detailed description, in view of the accompanying drawings, in which like reference characters refer to like parts throughout. In particular:

FIG. 1 is a medial side view of an embodiment of a stay configured for use in an ankle brace.

FIG. 2A and 2B depict medial and cross-sectional views, respectively, of an embodiment of the stay of FIG. 1, having a plurality of strengthening ribs.

FIG. 3 depicts a medial side view of an embodiment of a stay for use in an ankle brace.

FIG. 4 depicts a medial side view of the stay of FIG. 3 with additional strengthening ribs.

FIG. 5 depicts an embodiment of a stay for use in an ankle brace, the stay having a narrowing neck in an ankle region.

FIG. 6 depicts a front view of an ankle brace having an embodiment of a stay applied to the ankle in a loose configuration.

FIG. 7 depicts a front view of an ankle brace having an embodiment of a stay applied, in a tightened configuration.

### WRITTEN DESCRIPTION

Disclosed herein are ankle braces and methods of assembling and using such braces. The braces include one or more three-dimensional, reinforcing stays configured for stabilizing the ankle of a user against inversion and eversion. In certain embodiments, the ankle brace has an ankle support housing that is applied to the ankle of a user and can be tightened about the ankle. The one or more stays have a supporting upper plate, for example a plastic material, having anterior and posterior edges and a proximal region that can be applied to a user, extending longitudinally along the user's leg above the user's ankle. The ankle brace stay has an ankle region with an ankle-facing medial side and a non-ankle facing lateral side. The proximal region includes a contoured region that is disposed along the ankle-facing side to conform to the malleolus of the user's ankle. The contoured ankle region is conformable by heat molding (or other process).

In embodiments, the entire supporting upper plate (preferably the entire stay) is heat moldable and conforms to the patient's ankle and other lower leg areas, when warmed to at least about 165° F. In use, the stay is inserted into a pocket or sleeve or other compartment of an ankle brace, and the entire brace with the stay can be heated and then applied to the user's leg by straps or other fasteners. Fastening the heated brace to the user applies pressure to the brace, thereby molding and conforming it to the lower leg.

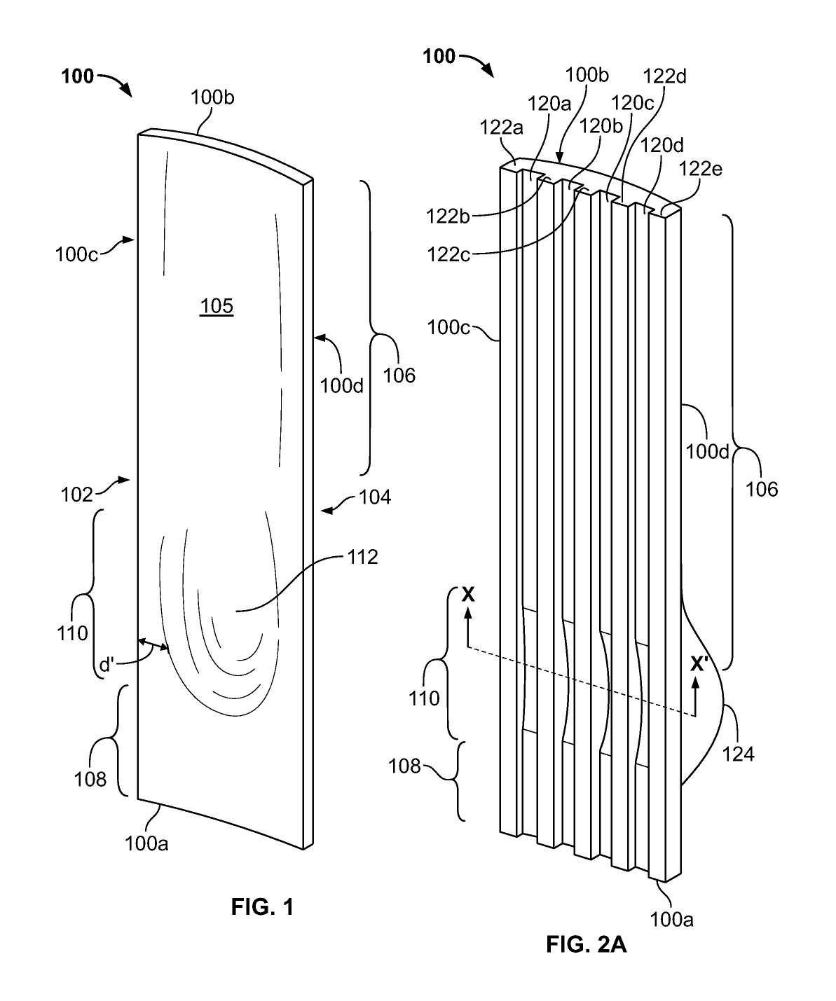

FIG. 1 shows a medial-side view of a stay 100 that can be used in an ankle brace to help support the ankle against inversion and eversion. As shown, the stay 100 includes a distal tip 100a and a proximal tip 100b, with a perimeter that has an anterior edge 100c and a posterior edge 100d disposed between those tips. The stay forms a supporting plate 105 for stabilizing an ankle. The stay 100 includes a proximal region 106, a distal region 108, and an ankle region 110 disposed between the proximal and distal regions. The proximal region 106 extends distally from the proximal tip 100b, and the distal region 108 extends proximally from the distal tip 100a. The stay 100 includes a medial facing side 102 and a lateral facing side 104.

As shown, the medial facing side 102 also includes the ankle region 110 having a contoured interior region 112 disposed within and along the ankle-facing medial side 102. The contoured region 112 can be preformed within the stay, prior to inserting it into a brace, so as to provide a general guide to placement on the ankle. For example, the contoured region 112 can have a cavity formed within the medial side 102 so as to align generally with the malleolus of an ankle. After the stay 100 is subsequently heat-molded, for example as described below, the ankle region and particularly the cavity or other contoured region 112 conforms to the specific user's ankle malleolus. The contoured region 112 is offset from the anterior edge 100c and posterior edge 100d by a distance of d′ shown on FIG. 1. Thus, the contoured region 112 is placed within the stay so as to align with the malleolus to support it, but without weakening the anterior or posterior edges of the stay.

The ankle region 110 is preferably formed of a thermo-formable polymer that is moldable at temperatures between about 150° F. and 250° F. but stiff at temperatures below approximately 150° F. The contoured region is thus moldable to fit the particular patient's malleolus. In certain implementations, the contoured region is heat formable so that it can be fit in real time to the patient, then stiffen as it cools for patient-specific fit. Examples of suitable materials for the stay include thermoplastic alloys formed from one or more polymers. Suitable polymers include polyester, polyetheylene, polyvinyl chloride, polyethylene tetraphthalate, polyamide, or PVC foam such as Sintra™ or Komatex™, or combinations thereof. An example of a suitable heat-formable material for the stay includes a thermo formable material provided by DJO Global under the trademark “Exos 40BX”. In certain embodiments, the entire stay is constructed of the thermo formable material so it can be heated then fit and molded to the user longitudinally along substantially the entire length of the stay 100. In some embodiments, the ankle region is a heat moldable insert that is plugged into a slot or open cavity of a stiff stay (e.g., any of the stays discussed above in the “Background”). Thus, in alternative embodiments, the stay can include permanently stiff regions with a heat formable ankle insert disposed between the stiff regions.

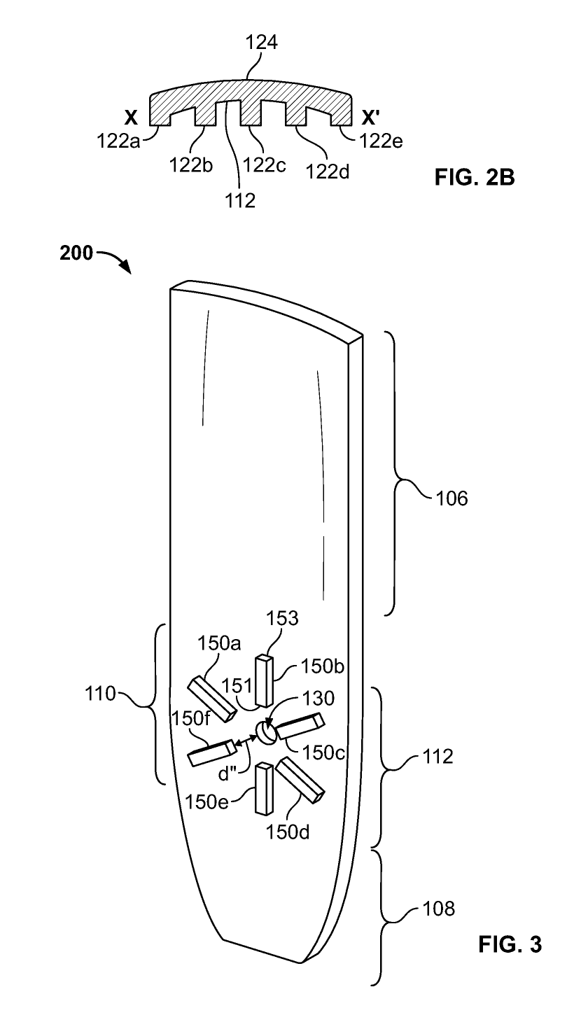

FIGS. 2A and 2B depict medial side and cross-sectional distal views, respectively, of a modification of stay 100 that includes stiffening elements provided for added resistance to inversion and eversion. The stiffening elements extend longitudinally along the medial (ankle-facing) surface of the stay. In particular, the stay 100 includes a plurality of stiffening ribs 122a-122e disposed longitudinally from the distal tip 100a to the proximal tip 100b. The stiffening ribs 122a-122e are separated by troughs 120a-120d. The ribs support the ankle against inversion and eversion, while the surrounding troughs provide flexibility to the ankle, allowing it to move in dorsi and plantar flexion, while stabilizing the ankle against inversion and eversion. In certain implementations, the stay with its ribs and troughs are injection molded in the three-dimension configuration. In alternative embodiments, the ribs and the troughs are die-cut from a pre-molded stay 100, leaving the plurality of ribs as co-molded portions of the original stay 100.

Also shown in FIGS. 2A and 2B, the stay 100 includes a convex region 124 that extends behind the ankle region 110. The contoured region 112 is encased within the convex region 124 for additional support. Also shown, the plurality of ribs 122a-122e extend longitudinally from the proximal region 106 through the ankle region 110 and through the distal region 108.

FIG. 2B is a cross-sectional view of FIG. 2A, taken along lines X-X′. As shown, the stiffening ribs 122a-122e extend through the contoured region 112. The components of the contoured region 112 of the ankle region 110 (including the troughs 120a-d and ribs 122a-e) are heat moldable to conform to the contours of the particular patient's malleolus. The strengthening ribs help resist inversion and eversion motion of the ankle, while the contoured region 112 provides comfort and improved fit.

FIG. 3 depicts an embodiment of a stay 200 for use in an ankle brace similar to the stay 100 shown in FIG. 1. The stay 200 includes a pre-formed hub and one or more spokes in the ankle region 110. In particular, the ankle region 110 provides pre-formed contouring by use of the series of spoke ribs 150a-150f disposed about the hub 130 that corresponds approximately to the center of the ankle region 110.

Each spoke 150 has a leading tip 151 and a trailing tip 153 that points radially toward the hub 130. The leading tip 151 is spaced away from the ankle center by a distance of d″. Each of the leading tips 151 of the respective ribs 150a-150f would, in use, abut the malleolus and support it as it rests within the contoured region 112 of the stay. In alternative embodiments, the leading tips of the respective ribs are joined together at the center hub or by an added ring (not shown) in a unitary web of spokes.

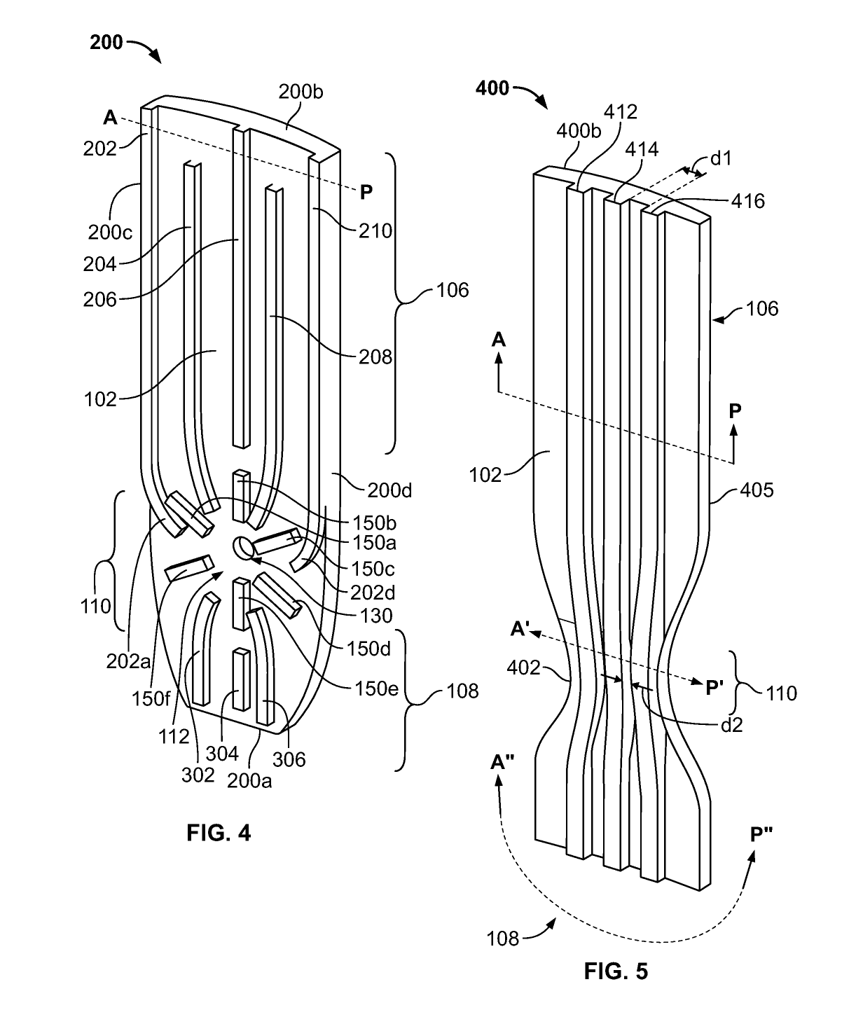

FIG. 4 depicts the stay 200 shown in FIG. 3 with strengthening ribs disposed longitudinally along proximal and distal regions 106 and 108. The ribs include proximal ribs 202, 204, 206, 208, and 210, and distal ribs 302, 304, and 306. As shown, the proximal rib 206 and distal rib 304 are generally straight and are disposed longitudinally along the midline, located approximately mid-way between anterior and posterior edges of the stay. The remaining ribs are disposed posterior or anterior to these midline ribs and are spaced apart. One or more of the ribs may be pre-contoured to conform to the user's leg in one or more areas above (region 106) and below (region 108) the ankle. For example, the remaining ribs can be shaped with arcs or other curves that align with ankle anatomy. Also shown, the ankle region ribs are spaced away from the longitudinal strengthening ribs to help provide additional inversion and eversion control of the patient's ankle.

The anterior and posterior strengthening ribs 202 and 210 are disposed coextensively along posterior and anterior edges 200c and 200d of the stay 200. Ribs 204-208 are disposed on the medial face 102 and extend between the anterior and posterior edges. These ribs 202-210 extend above (proximal to) the ankle region 110 and have distal tips (see e.g., 202a and 202d) that extend into the ankle region and terminate in alignment with or above the center hub 130. The distal ribs 302-306 are also spaced away from the anterior and posterior edges 200c and 200d and extend upwardly from distal tip 200a into the ankle region 110, terminating at or below the center hub 130. As further shown in FIG. 4, the ribs and spokes are spaced away from each other in the ankle region 110 (see e.g., 202d and 150d), which provides a contoured shape that can adapt to the malleolus and surrounding ankle structure and help facilitate a conforming fit to the ankle.

The stay, including its components (e.g., ribs and spokes), is heat moldable. In use, the stay is placed in an ankle brace housing and heated to a temperature at or above 150° F. and less than 250° F., then placed on a user's ankle and strapped, laced or otherwise mechanically fastened to the ankle. The fastening tightens and molds the heated stay to the user's ankle for fit and stability. The brace then stiffens as it cools.

The perimeter of the stay and the alignment of the longitudinal ribs along the support plate can be structured to permit plantar and dorsi flexion while still supporting the ankle against inversion and eversion. This is done, in some embodiments, by reducing the medial-lateral thickness of the support plate. In some embodiments, the support plate (e.g., plate 105) is less than 0.06 inches thick. In some embodiments the thickness of the support plate (e.g., plate 105) is between about 0.03 inches and about 0.06 inches thick. Traditional stays typically are not able to provide anterior-posterior flexing while still maintaining inversion and eversion control. Instead, the traditional stays require a thicker material in order to provide sufficient support against inversion and eversion. The heat-moldable stays discussed herein can provide similar support to the traditional stays, but do so with a thinner material for added anterior-posterior flexion with added comfort.

In certain implementations, medial-lateral stiffness and anterior-posterior stiffness is customized and selected to provide an appropriate level of support for the user. One application is made by varying the anterior-posterior width of the support plate between the proximal and distal ends of the stay, providing a narrow region for added flexibility, with stiffeners for inversion/eversion control.

FIG. 5 illustrates an embodiment that may accomplish customization. The stay 400 is structured to fit within a pocket, sleeve or other compartment of an ankle brace. The stay 400 is heat moldable and provides a support plate 405 with a series of longitudinally extending ribs 412-416 disposed along the medial face 102 of the stay 400. Portions of the ribs 412-416 pass through the ankle region 110 and stiffen the stay against medial-lateral bending (and, therefore, stabilize the ankle against inversion and eversion movement), while the neck 402 facilitates plantar and dorsi flexion, similar to the stays discussed above. The plate 405 also has a narrow neck 402 in the ankle region 110 that supports the ankle of the user. The neck 402 in the ankle region 110 allows the brace to flex in the anterior-posterior direction in the region of the neck for improved flexion.

In particular, the overall narrowing of the plate 405 in the neck 402 provides a flexion zone about which the narrow neck can flex in the anterior-posterior direction (along the A′-P′ direction arrow shown in FIG. 5). The distal region 108 can also flex angularly beneath the ankle region 110 about that flexion zone along the A″-P″ direction arrow shown in FIG. 5. In certain embodiments, the reduced stiffness allows the flexion zone to function like a hinge about which the distal region 108 can flex in the A-P direction. In particular, the reduced stiffness of the stay 400 reduces the area moment of inertia of the ankle region 110 (in the posterior-anterior direction A-P). Because the support plate 405 is narrower in the region of the neck 402 (e.g., the ankle region 110) than in the proximal region 106 or distal region 108, the plate 405 is more flexible in the A-P direction in that neck region 402 and therefore can support the flexing of the distal region 108 along one or both of the direction arrows A′-P′ and A″-P″. The flexing of the distal region 108 allows the user to flex the ankle in dorsi or plantar flexion.

The flexion of the flexion zone can be enhanced by the converged spacing of the ribs in the ankle region 110. As shown, the ribs 412-416 run through the neck region 402 to provide resistance against medial-lateral flexion (to impede inversion and eversion), while the narrow neck allows flexion in the anterior-posterior direction. In the ankle region 110, the ribs 412-416 converge toward each other so that they are closer together in the neck region 402 than they are in the proximal 106 or distal 108 regions. As shown, the adjacent ribs 412 and 414 are spaced apart by distance d₁ in the proximal region but only by distance d₂ in the ankle region 110, which is less than distance d₁. The distal region 108 of the stay flanges outwardly in the anterior-posterior direction below the neck to support the calcaneus. In that distal region 108, the ribs flare outwardly again and return to their original spacing of d₁. The converged spacing of the ribs in the ankle region 110, so they are closer together, would reduce the area moment of inertia in the ankle region 110. The area moment of inertia is driven exponentially by additions or reductions of material along the width of the stay 400 (in the A-P direction). Reducing the area moment of inertia would reduce the stiffness in the ankle region 110 to permit natural movement in the A-P direction. Thus, the stay 400 can provide support against inversion and eversion to protect a wearer's ankle, while allowing natural movement of the ankle to provide comfort to the wearer.

The flexibility of the neck region 402 can also be enhanced in the A-P direction by using a different number of longitudinal ribs in the ankle region 110 than the number of ribs used in the proximal region 106 or distal region 108. In the example of FIG. 5, the stiffness of the stay 400 can be altered and customized by using fewer (or more) ribs in the ankle region 110 than are included in the proximal region 108. For example, one or even two of the ribs 412-416 can terminate in a position along the medial surface that is above (proximal to) the ankle region 110, leaving only two or even only one rib (e.g., rib 414) to extend through the ankle region 110. In such implementations, rib 412 (and also rib 416 if more than one rib is omitted or removed) stops before reaching the ankle region 110. In the embodiment of FIG. 5, where the ribs are generally rectangular, omitting one rib of the three (e.g., by not extending it into the ankle region 110) would reduce the stiffness of the stay 400 in the medial-lateral direction by approximately ⅓ (by removing one of three ribs). But omitting one rib of three and concurrently reducing the width of the neck region can more dramatically reduce the stiffness of the stay 400 in the anterior-posterior direction and thus allow more dorsi/plantar flexion. That reduction occurs because the area moment of inertia is driven exponentially by additions or reductions of material along the width of the stay 400 (in the A-P direction). Reducing the ribs will exponentially reduce the area moment of inertia along that width.

In some embodiments, no ribs are included in the ankle region, which alters the stiffness in that region even further. Altering the number of ribs that pass through the ankle region 110 can alter the relative medial-later and anterior-posterior stiffness of the stay while the strengthening ribs 412-416 provide strengthening support against inversion and eversion. Thus the stiffening structures disclosed herein can impede inversion and eversion yet allow plantar and dorsiflexion in a way that provides comfort and better fit to the user.

The stays discussed herein are inserted into an ankle brace that is worn by a user. Non-limiting examples of suitable braces including a lace-up brace with a soft goods housing (e.g., non-stretch Nylon housing), such as the DonJoy Stabilizing Pro Ankle Brace or the RocketSoc lace-up, sold by DJO Global.

In use, the stay is inserted into a compartment, such as a sleeve or pocket, of the brace housing to form the ankle brace. The ankle brace is then heated to a predetermined temperature or with a predetermined heat load, for example between about 150° F. and 250° F. The heated brace is then applied to the user's ankle and laced or strapped to the appropriate fit. While heated, the moldable stay within the brace housing changes shape and conforms to the particular contours of the user's ankle. Any pre-formed crevices or cavities, strengthening ribs or spokes, or other surface features included in the stay are also heat moldable and also conform to the ankle as the straps and laces are pulled tight. As the brace and stay cool, the stay stiffens in the conformed configuration about the ankle, providing a stabilizing ankle support structure that is specific to that ankle. The stay can be removed and re-heated for repeated use, or for re-shaping and adjusting the configuration (for example to accommodate changes in swelling in the ankle).

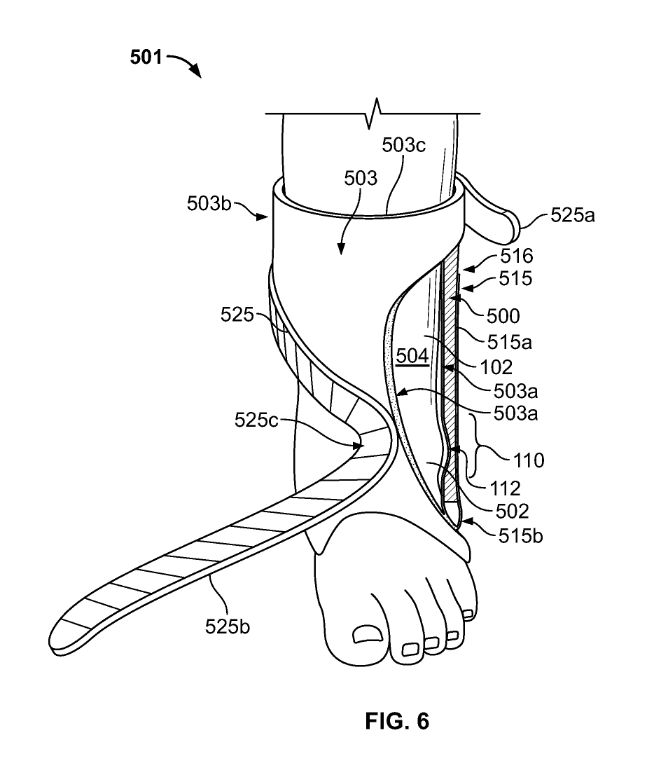

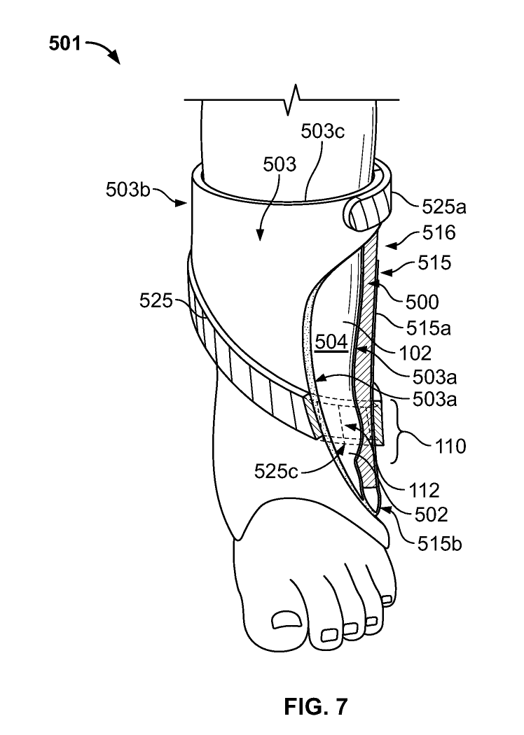

FIGS. 6-7 depict cut-away views of an embodiment of an ankle brace 501 and a stay 500 disposed within the ankle brace in a loose configuration (FIG. 6) and in a tightened configuration (FIG. 7). The stay 500 is similar to the other stays disclosed herein and could be any of such stays. The ankle brace 501 has an ankle support housing 503 applied to the ankle 504 of the user. The housing 503 has a proximal opening 503c that receives the user's ankle, an inner layer 503a that contacts the user's ankle after the ankle is seated within the housing 503, and an outer layer 503b that is exposed to the ambient environment. The inner layer 503a is thin (e.g., flexible nylon) and fits along the lateral side 504 of the ankle, including the lateral malleolus 502. As shown, the stay 500 includes a support plate with a medial face 102 that aligns with the user's lower leg, in this case the lateral side 504 of the leg. The stay also has an ankle region 110 with a contoured interior cavity 112 disposed about the lateral side of the malleolus 502 of the user and that can conform to the malleolus and surrounding ankle region in a conformable, fitted manner.

As shown, the stay 500 is disposed within a pocket 515 formed on the lateral side of the brace housing 503. The pocket 515 includes an outer fabric layer 515a that is stitched or otherwise attached to the inner layer 503a of the brace housing 503 in the distal region 515c. An opening 516 is defined between the proximal end of the outer fabric layer 515a and the inner layer 503a of the housing 503. The opening 516 receives the stay 500, so that the stay 500 is disposed between the outer fabric layer 515a and the inner layer 503a of the housing.

A tightening strap 525 is also included. The strap 525 has a proximal end 525a, a distal end 525b, and an intermediate region 525c. The distal end 525b and proximal end 525a anchor to the housing 503. In use, in the tightened configuration of FIG. 7, the strap 525 is wrapped around the housing 503, with the intermediate region 525c extending around the lateral side 504 of the housing and across the stay 500. Tightening the strap 525 actuates the strap to tighten the brace about the ankle from the loose state (FIG. 6) to the tightened state (FIG. 7) about the ankle. Tightening the brace as in FIG. 7 tightens the heated stay 500 disposed therein so the stay conforms to the ankle. Thus, in the loose state the stay is in a first shape configuration, but that configuration changes to a second shape when the stay is tightened. For example, as shown in FIG. 6, the stay may be relatively flat or slightly arced when in the loose state, but upon tightening it conforms to mirror the shape of the ankle region, including its various external contours. Upon cooling in ambient air (while in the tightened state), the stay stiffens to provide contoured, patient-specific support about the ankle to impede inversion and eversion.

Similar applications can be made to the medial side of the ankle by using a medial pocket or sleeve on the medial side of the housing 503 and ensuring that the strap 525 extends around the medial side so as to actuate the tightening mechanism, as discussed above. Also, other compartments in the brace may be used, in lieu of the pocket, to hold the stay. For example, a sleeve with a fully sewn or attached perimeter may also be substituted for the pocket.

The stays discussed herein are preferably formed by injection molding using a 3-dimensional mold that accommodates medial-lateral thickness, anterior-posterior width, and pre-shaped contouring along the medial face, particularly in the ankle region. The molds can also include slots for forming one or more ribs and troughs, such as those discussed above. In alternative configurations, the stays are machined or die-cut.

Variations and modifications will occur to those of skill in the art after reviewing this disclosure. The disclosed features may be implemented, in any combination and sub-combination (including multiple dependent combinations and sub-combinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited are hereby incorporated by reference herein in their entireties and made part of this application.

1. An ankle brace, comprising:
a housing having an opening for receiving a user's ankle, lateral and medial sides that fit around the ankle, and a compartment on at least one of the lateral and medial sides; and
a stay sized to fit within the compartment; the stay further comprising:
a support plate having anterior and posterior edges, a distal region, and a proximal region that, when applied to a user, extends longitudinally along the user's leg above the user's ankle; and
a heat-moldable ankle region disposed longitudinally between the proximal and distal regions of the support plate, the heat-moldable ankle region having an ankle facing side, a non-ankle facing side, and a contoured region that is disposed along the ankle facing side and is positioned on the ankle-facing side so as to align with the ankle's malleolus.

2. The ankle brace stay of claim 1, comprising at least one tightening strap that is anchorable to the housing and actuatable to tighten the brace about the ankle from a loose state to a tightened state.

3. The ankle brace of claim 2, wherein the ankle region is a mid-region of the support plate that is co-molded to and unitary with the proximal and distal regions.

4. The ankle brace of claim 3, wherein the stay in the loose state is in a first shape configuration and the stay in the tightened state is in a second shape configuration that differs from the first shape configuration.

5. The ankle brace of claim 3, wherein at least one of the anterior and posterior edges forms an anterior-posterior neck in the mid-region.

6. The ankle brace of claim 3, wherein the contoured region comprises a plurality of ribs.

7. The ankle brace of claim 6, wherein the ankle region comprises a hub, and each of the plurality of ribs extends radially as a spoke about the hub.

8. The ankle brace of claim 7, wherein the first plurality of ribs project medially from the ankle-facing side.

9. The ankle brace of claim 3, wherein the contoured region comprises a depression formed within the ankle-facing side prior to insertion of the stay in the pocket or sleeve.

10. The ankle brace of claim 3, wherein the support plate and ankle region are co-molded and heat moldable.

11. The ankle brace of claim 3, wherein the support plate includes a first plurality of heat-moldable ribs that extend longitudinally between the anterior and posterior edges and proximal to the ankle region.

12. The ankle brace of claim 11, wherein at least one of the first plurality of ribs extends longitudinally from a distal tip of the support plate to a proximal tip of the support plate.

13. The ankle brace of claim 11, wherein at least one of the first plurality of ribs extends longitudinally through the ankle region.

14. The ankle brace of claim 11, comprising a plurality of troughs within the medial side of the plate, the plurality of troughs extending longitudinally along the support plate, and at least one of the first plurality of ribs is disposed between adjacent troughs.

15. The ankle brace of claim 14, wherein the plurality of troughs extend longitudinally through the ankle region.

16. The ankle brace of claim 15, wherein the distal end of each rib of the first plurality of ribs terminates within or proximal to the contoured region.

17. The ankle brace of claim 16, comprising a second plurality of ribs disposed on a medial side of the ankle region and forming a series of spokes around a hub in the ankle region, and at least one rib of the first plurality of ribs is spaced between adjacent ribs of the second plurality of ribs.

18. The ankle brace of claim 11, wherein at least one each of the first plurality of ribs extends longitudinally along the ankle region and the distal region of the plate.

19. The ankle brace of claim 18, comprising adjacent ribs of the first plurality of ribs being spaced apart at respective proximal ends by a first distance, and respective sections of those adjacent ribs being spaced apart within the ankle region by a second distance.

20. The ankle brace of claim 19, wherein the first distance is greater than the second distance.

21. The ankle brace of claim 11, comprising a second plurality of ribs that extend longitudinally through the ankle region, the second plurality being fewer than the first plurality of ribs.

22. The ankle brace of claim 21, comprising one rib that extends longitudinally through the ankle region.

23. The ankle brace of claim 11, wherein the contoured region has a perimeter edge that is spaced between the anterior and posterior edges of the support plate.

24. The ankle brace of claim 11, comprising a flange in the mid-region formed from at least one of anterior and posterior edges.

25. The ankle brace of claim 11, wherein the compartment is a pocket disposed on a side of the brace housing, the pocket having a proximal opening, and the stay is disposed within the pocket.

26. The ankle brace of claim 11, wherein the stay has an average thickness of between about 0.03 inches and about 0.06 inches.

27. A method of assembling an ankle brace, comprising:
forming a pocket in a medial or lateral side of a flexible ankle housing, the pocket having an opening,
forming a heat moldable stay having a width that fits within the opening, and
inserting the stay into the opening of the pocket.

28. A method of treating an ankle injury, comprising steps of:
inserting a heat moldable stay into a pocket of an ankle brace,
heating the stay-containing ankle brace to a temperature between about 150° F. and 250° F.,
inserting the ankle into the heated brace and aligning the malleolus of the ankle with a medial side of the inserted stay, and
conforming the heated stay to contours of the ankle by tightening one or more straps of the ankle brace to the ankle.
